SEQUENCE LISTING (12 sequences)

SEQ ID NO: 1 - protein, 253 aa, Artificial sequence
  EP0751221 recombinant Protein
XYERXRLRVTHQTTGXEYFRFITLLRDYVSSGSFSNEIPLLRQSTIPVSDAQRFVLVELTNQGGDSITAAIDVTNLYVVAYQAGDQSYFLRDAPRGAETHLFTGTTRSSLPFNGSYPDLERYAGHRDQIPLGIDQLIQSVTALRFPGGSTRTQARSILILIQMISEAARFNPILWRARQYINSGASFLPDVYMLELETSWGQQSTQVQHSTDGVFNNPIRLAIPPGNFVTLTNVRDVIASLAIMLFVCGERPS

SEQ ID NO: 2 - protein, 256 aa, Artificial sequence
  EP1051495 recombinant Protein
XYERLRLRVTHQTTGXEYFRFITLLRDYVSSGSFSNEIPLLRQSTIPVSDAQRFVLVELTNQGXDSXTAAIDVTNXYVVAYQAGDQSYFLRDAPRGAETHLFTGTTRXSSLPFXGSYXDLERYAGHRDQIPLGIXQLIQSVXALRXPGGSTRXQARSILILIQMISEAARFNPILWRXRQXINSGXSFLPDXYMLELETSWGQQSTQVQHSTDGVFNNPXRLAIXXGNFVTLXNVRXVIASLAIMLFVCGERPSXX

SEQ ID NO: 3 - protein, 257 aa, Artificial sequence
  EP1051495 recombinant Protein
XYERLRLRVTHQTTGDEYFRFITLLRDYVSSGSFSNEIPLLRQSTIPVSDAQRFVLVELTNQGQDSITAAIDVTNAYVVAYQAGDQSYFLRDAPRGAETHLFTGTTRDRSSLPFTGSYTDLERYAGHRDQIPLGIEQLIQSVSALRYPGGSTRAQARSILILIQMISEAARFNPILWRYRQDINSGESFLPDMYMLELETSWGQQSTQVQHSTDGVFNNPFRLAISTGNFVTLSNVRSVIASLAIMLFVCGERPSSS

SEQ ID NO: 4 - protein, 264 aa, Artificial sequence
  EP0751221 recombinant Protein
XDDVTCSASEPTVRIVGRNGMCVDVRDDDFRDGNQIQLWPSKSNNDPNQLWTIKRDGTIRSNGSCLTTYGYTAGVYVMIFDCNTAVREATLWQIWGNGTIINPRSNLVLAASSGIKGTTLTVQTLDYTLGQGWLAGNDTAPREVTIYGFRDLCMESNGGSVWVETCVSSQKNQRWALYGDGSIRPKQNQDQCLTCGRDSVSTVINIVSCSAGSSGQRWVFTNEGAILNLKNGLAMDVAQANPKLRRIIIYPATGKPNQMWLPVP

SEQ ID NO: 5 - protein, 268 aa, Artificial sequence
  EP0751221 recombinant Protein
XDDVTCSASEPTVRIVGRNGMCVDVRDDDFRDGNQIQLWPSKSNNDPNQLWTIKRDGTIRSNGSCLTTYGYTAGVYVMIFDCNTAVREATLWQIWGNGTIINPRSNLVLAASSGIKGTTLTVQTLDYTLGQGWLAGNDTAPREVTIYGFRDLCMESNGGSVWVETCVSSQKNQRWALYGDGSIRPKQNQDQCLTCGRDSVSTVINIVSCSAGSSGQRWVFTNEGAILNLKNGLAMDVAQANPKLRRIIIYPATGKPNQMWLPVPGGYH

SEQ ID NO: 6 - protein, 265 aa, Artificial sequence
  EP1051495 recombinant Protein
XDDVTCSASEPTVRIVGRXGMXVDVRDDDFHDGNQIQLWPSKSNNDPNQLWTIKRDXTIRSNGSCLTTYGYTAGVYVMIFDCNTAVREATIWQIWXNGTIINPRSNLVLAASSGIKGTTLTVQTLDYTLGQGWLAGNDTAPREVTIYGFRDLCMESNXGSVWVETCXSSQXNQXXWALYGDGSIRPKQNQDQCLTXGRDSVSTVINIVSCSXXSXXQRWVFTNEXAILNLKXXXXXDVAQANPKLRRIIIYPATGKPNQMWLPVX

SEQ ID NO: 7 - protein, 264 aa, Artificial sequence
  EP1051495 recombinant Protein
XDDVTCSASEPTVRIVGRNGMCVDVRDDDFHDGNQIQLWPSKSNNDPNQLWTIKRDGTIRSNGSCLTTYGYTAGVYVMIFDCNTAVREATIWQIWGNGTIINPRSNLVLAASSGIKGTTLTVQTLDYTLGQGWLAGNDTAPREVTIYGFRDLCMESNGGSVWVETCVSSQQNQRWALYGDGSIRPKQNQDQCLTCGRDSVSTVINIVSCSAGSSGQRWVFTNEGAILNLKNGLAMDVAQANPKLRRIIIYPATGKPNQMWLPVP

SEQ ID NO: 8 - protein, 265 aa, Artificial sequence
  EP1051495 recombinant Protein
XDDVTCSASEPTVRIVGRNGMRVDVRDDDFHDGNQIQLWPSKSNNDPNQLWTIKRDGTIRSNGSCLTTYGYTAGVYVMIFDCNTAVREATIWQIWDNGTIINPRSNLVLAASSGIKGTTLTVQTLDYTLGQGWLAGNDTAPREVTIYGFRDLCMESNGGSVWVETCDSSQKNQGKWALYGDGSIRPKQNQDQCLTSGRDSVSTVINIVSCSGASGSQRWVFTNEGAILNLKNGLAMDVAQANPKLRRIIIYPATGKPNQMWLPVF

SEQ ID NO: 9 - protein, 265 aa, Artificial sequence
  EP1051495 recombinant Protein
XDDVTCSASEPTVRIVGRSGMRVDVRDDDFHDGNQIQLWPSKSNNDPNQLWTIKRDNTIRSNGSCLTTYGYTAGVYVMIFDCNTAVREATIWQIWDNGTIINPRSNLVLAASSGIKGTTLTVQTLDYTLGQGWLAGNDTAPREVTIYGFRDLCMESNQGSVWVETCDSSQKNQGKWALYGDGSIRPKQNQDQCLTVGRDSVSTVINIVSCSGASGSQRWVFTNEYAILNLKSGLAMDVAQANPKLRRIIIYPATGKPNQMWLPVF

SEQ ID NO: 10 - protein, 265 aa, Artificial sequence
  EP1051495 recombinant Protein
XDDVTCSASEPTVRIVGRNGMRVDVRDDDFHDGNQIQLWPSKSNNDPNQLWTIKRDGTIRSNGSCLTTYGYTAGVYVMIFDCNTAVREATIWQIWDNGTIINPRSNLVLAASSGIKGTTLTVQTLDYTLGQGWLAGNDTAPREVTIYGFRDLCMESNGGSVWVETCDSSQKNQGKWALYGDGSIRPKQNQDQCLTSGRDSVSTVINIVSCSGASGSQRWVFTNEGAILNLKTGLAMDVAQANPKLRRIIIYPATGKPNQMWLPVF

SEQ ID NO: 11 - protein, 265 aa, Artificial sequence
  EP1051495 recombinant Protein
XDDVTCSASEPTVRIVGRNGMRVDVRDDDFHDGNQIQLWPSKSNNDPNQLWTIKRDGTIRSNGSCLTTYGYTAGVYVMIFDCNTAVREATIWQIWDNGTIINPRSNLVLAASSGIKGTTLTVQTLDYTLGQGWLAGNDTAPREVTIYGFRDLCMESNGGSVWVETCDSSQKNQGKWALYGDGSIRPKQNQDQCLTSGRDSVSTVINIVSCSGASGSQRWVFTNEGAILNLKKGPAMDVAQANPKLRRIIIYPATGKPNQMWLPVF

SEQ ID NO: 12 - protein, 265 aa, Artificial sequence
  EP1051495 recombinant Protein
XDDVTCSASEPTVRIVGRNGMRVDVRDDDFHDGNQIQLWPSKSNNDPNQLWTIKRDGTIRSNGSCLTTYGYTAGVYVMIFDCNTAVREATIWQIWDNGTIINPRSNLVLAASSGIKGTTLTVQTLDYTLGQGWLAGNDTAPREVTIYGFRDLCMESNGGSVWVETCDSSQKNQGKWALYGDGSIRPKQNQDQCLTSGRDSVSTVINIVSCSGASGSQRWVFTNEGAILNLKNSLMVDVAQANPKLRRIIIYPATGKPNQMWLPVF

(19) United States
(12) Patent Application Publication
(10) Pub. No.: US 2014/0220076 A1
(43) Pub. Date: Aug. 7, 2014

(54) Recombinant Mistletoe Lectin and use Thereof as an Adjuvant

(71) Applicants: Klaus Witthohn, Overath (DE); Hans Lentzen, Rosrath (DE)

(72) Inventors: Klaus Witthohn, Overath (DE); Hans Lentzen, Rosrath (DE)

(73) Assignee: Cytavis Biopharma GmbH, Hamburg (DE)

(21) Appl. No.: 14/129,441

(22) PCT Filed: Jun. 27, 2012
(86) PCT No.: PCT/EP2012/062521

§ 371 Date: Feb. 11, 2014

(30) Foreign Application Priority Data
Jun. 27, 2011 (DE) 10 2011 115 023.4

(51) Int. Cl. A61K 39/39

(52) U.S. Cl. CPC A61K 39/39; A61K 2039/55516
USPC 424/227.1; 424/184.1

(57) ABSTRACT

The invention relates to vaccines that comprise antigens and an adjuvant, and to the use of the adjuvant, wherein the adjuvant is selected from a recombinant mistletoe lectin.

The invention relates to vaccines that comprise antigens and an adjuvant, and to the use of the adjuvant, wherein the adjuvant is selected from a recombinant mistletoe lectin.

In many diseases, vaccines are only partially or not at all effective. Research into protective immunity and adjuvants that induce strong immune responses can help to generate effective vaccines against pathogens (McKee A S, MacLeod M K L, Kappler J W et al. 2010 BMC Biology; 8: 37-46). Vaccines are vaccines made of live, attenuated (i.e. having weakened virulence) or inactivated pathogenic agents or inactivated (detoxified) toxins or toxoids of pathogens or fragments of the surface structure of pathogens. Currently a formal distinction is made between three main types of vaccines: a.) Attenuated live vaccines, which comprise a virus or a bacterium that is similar to but less pathogenic than the actual pathogen; b.) inactivated vaccines that are heat-inactivated or chemically inactivated particles of the pathogen; or c.) subunit vaccines, which are made from components of the pathogen.

In addition to an antigen that is the target of the adaptive immune response, vaccines usually also contain either pathogen-associated molecular patterns (PAMPs) or other substances that strengthen or affect the adaptive response. These substances are described as adjuvants (McKee et al 2010 (supra)).

A known adjuvant that has a long history of use in human vaccines is aluminum salt (also referred to as alum). Proteins (antigens) of the pathogen are adsorbed onto the aluminum salt, wherein a suspension is produced which is injected intramuscularly.

Hepatitis B is an infectious disease of the liver with the hepatitis B virus (HBV), which often follows an acute course (90%) and occasionally follows a chronic course. Approximately 350 million people are chronically infected with hepatitis B, making this the most common viral infection worldwide. Chronic inflammation of the liver can lead to hepatic cirrhosis and to hepatocellular carcinoma. Treatment of chronic hepatitis B is difficult and, therefore, preventive vaccination is the most important measure for preventing infection and reducing the number of virus carriers.

Various hepatitis B vaccines are commercially available, such as Engerix-B from GlaxoSmithKline. In this vaccine, aluminum hydroxide is used as the adjuvant, and the hepatitis B surface antigen HBsAg is produced using recombinant DNA technology in yeast cells (Saccharomyces cerevisiae).

In a cancer treatment by means of vaccination, the vaccines can be therapeutic or prophylactic. Adjuvants are administered in order to strengthen the efficacy of prophylactic or therapeutic vaccines, i.e. to induce a strong and sustained immune response. There is a need for subunit vaccines in particular. Subunit vaccines are made of purified antigens that are specifically recognized by lymphocytes. Although they are safer than whole-organism vaccines, they are incapable alone of optimally activating the immune system, because they lack intrinsic PAMPs (McKee et al 2010 (supra)). Adjuvants can affect the balance of the induced antibody- and cell-mediated immunity. The use of such substances therefore makes it possible to reduce the dose of an antigen and the number of injections required (Salk J E, Laurent A M & Bailey M L 1951 Am J Public Health Nations Health 41: 669-77).

Adjuvants are also subdivided into two categories: Carrier systems and “immune potentiators”, i.e. immune boosters (Pashine A, Valiante N M & Ulmer J B 2005 Nature medicine 11: 63-68, Pichichero M E 2008 Human vaccines 4(4): 262 270). Carrier-based adjuvants (aluminium) increase the interaction between vaccine components and key cells of the immune system.

Immune boosters directly activate antigen-presenting cells (APCs) and innate immune responses by the use of specific receptors [e.g. Toll-like receptors (TLRs)] (Pashine et al. (supra), O'Hagan D T & Valiante N M 2003 Nature reviews 2: 727-735).

Several types of vaccine adjuvants have been available since 1970, but to date only a few have been approved for use in humans (O'Hagan D T & De Gregorio E 2009 Drug Discov Today 14(11-12): 541-551).

Recently, new adjuvants such as Toll-like receptor (TLR) agonists and new particulate carrier systems, such as peptide-based vaccines, offer an option for modern immunotherapy.

New adjuvants are still necessary, however, which should have the following properties (Brunner R, Jensen-Jarolim E & Pali-Scholl 12010 Immunol Lett 128(1): 29-35):
a.) strengthening of Th17 cells. The adjuvants are characterized by the production of IL-17. They are an important modulator in inflammation and CD4+ T cell memory.
b.) inducing strong cellular responses, including T helper (Th) 1 cells and cytotoxic T lymphocytes (CTLs), in addition to antibodies (Guy B 2007 Nat Rev Microbiol 5: 505-517).

In general, adjuvants have different mechanisms of action (FIG. 1). Non-specific adjuvants (e.g. aluminum hydroxide) strengthen the antigen presentation by activation of the inflammasome pathway, which is characterized by secretion of IL-1β (Lamine Mbow M, De Gregorio E, Valianteal N M et al. 2010 Current Opinion in Immunology 2010, 22: 411-416). Aluminum forms a depot at the injection site, which results in a high local antigen concentration and therefore improves the uptake by antigen-presenting cells (APCs) (HogenEsch H 2002 Vaccine 20 (Suppl 3): 34-39). Furthermore, antigen detection can be accelerated by means of direct stimulation of immune cells (Gupta R K, Rost B E, Relyveld E et al 1995 Pharm Biotechnol 6: 229-248). These so-called type B adjuvants interact with APCs and antigens in a non-specific manner, and the effect thereof is based on a strengthening of the antigen-presenting MHC molecules.

In contrast to type B adjuvants, type A adjuvants have a specific mechanism of action. The majority of recently developed type A adjuvants, such as monophosphoryl lipid A (MPL), are specific agonists for TLRs. They act primarily on TLR and act indirectly by means of activation of APCs and triggering the secretion of cytokines, such as IL-12. In addition, TLR agonists can act on MHC receptors by means of an effective presentation of the administered antigens (Guy B 2007 (supra)).

Furthermore, type C adjuvants are described. The operation thereof is based on a strengthening of MHC receptors by interaction with co-stimulatory molecules on APCs. Efforts are underway to introduce type C adjuvants into clinical application. One known example is TGN1412, a new superagonistic anti-CD28 monoclonal antibody that directly stimulates T cells, although, in one study, cardiovascular shock and acute lung failure occurred due to a cytokine storm (Suntharalingam G, Perry M R, Ward S et al. 2006 N Engl J Med 355: 1018-1028).

In the prior art there is a strong demand for new, suitable adjuvants.

Plant-derived mistletoe extracts have been used for therapeutic purposes for centuries. Mistletoe preparations have been used with varying degrees of success in cancer therapy in particular (Bocci V 1993 J Biol Regulators and Homeostatic Agents 7(1): 1-6; Gabius H-J, Gabius S, Joshi S S et al. 1993 Planta Med 60: 2-7; Gabius H-J & Gabius S 1994 PZ 139: 9-16; Ganguly C & Das S 1994 Chemotherapy 40: 272-278, Hajto T, Hostanska K, Gabius H_J 1989 Cancer Res 49: 4803-4808, Hajto T, Hostanska K, Frei K et al. 1990 Cancer Res. 50: 3322-3326). It has been shown that the therapeutic effects are attributable to so-called mistletoe lectins (viscumin, Viscum album agglutinin, VAA) in particular. Mistletoe lectins have a cytotoxic effect and induce an unspecific immunostimulation, the positive effects of which are used to treat tumor patients. Various investigations involving mistletoe lectins in vitro (Hajto et al., 1990 (supra); Männel D N, Becker H, Gundt A et al. 1991 Cancer Immunol Immunother 33: 177-182; Beuth J, Ko K L, Tunggal L et al. 1993 Drug Res 43: 166-169) and in vivo (Hajto T 1986 Oncology 43 suppl 1: 51-65; Hajto et al., 1989 (supra), Beuth J, Ko H L, Gabius H-J et al. 1991 In Vivo 5: 29-32; Beuth J, Ko H L, Gabius H-J et al. 1992 J Clin Invest 70: 658-661), and clinical studies (Beuth et al., 1992 (supra)) showed an increased release of inflammatory cytokines (TNF-alpha, IL-1, IL-6) and an activation of cellular components of the immune system (TH cells, NK cells).

Analysis of mistletoe extract have so far resulted in the identification of three mistletoe lectins (ML-I, ML-II, ML-III) having different molecular weights and sugar-binding specificities. It was shown that the immunostimulating effect of mistletoe extract is attributable to ML-I. The ML-I lectin consists of two A- and two B-chains (MLA and MLB, respectively), each glycosylated. The A chain is responsible for an enzymatic inactivation of ribosomes (Endo Y, Tsurugi K & Franz H 1988 FEBS Lett 231: 378-380), while the B chain participates in carbohydrate binding. The two chains are linked together via disulphide bridges. The resulting mistletoe lectin monomers can associate into dimers with the formation of non-covalent bonds.

It is also possible to produce the biologically active mistletoe lectin using recombinant technology. EP 0751221 describes the isolation of mistletoe lectin polypeptides as a structurally homogeneous substance, wherein, proceeding from the genetic sequences of mistletoe lectin, recombinant, highly pure single chains (A-chain, B-chain) are produced, which can be reassociated in vitro and thereby yield a recombinant mistletoe lectin holoprotein, which, particularly advantageously, is protein-chemically, enzymatically and structurally homogeneous, so-called Aviscumine. According to EP 0751221, the recombinant mistletoe lectin polypeptide is suitable for therapeutic use as a holoprotein, a subchain, and in the form of subfragments, and is covered according to the invention. Biologically active mistletoe lectin can be produced by recombinant technology in E. coli and is referred to not only as Aviscumine, but also as “rViscumin” or “rML” (Eck J, Langer, M, Möckel, B et al 1999; Eur J Biochem 264: 775-784).

Although EP 0751221 does mention that recombinant mistletoe lectins would be feasible for the treatment of infectious diseases, no indication is disclosed that recombinant mistletoe lectin can be used as an adjuvant or in a vaccine.

Moreover, Lavelle et al (Lavelle E C, Grant G, Pusztal A et al. 2002. Immunology 107:268-274) describe the use of plant-derived lectins as adjuvants, which are administered mucosally by inhalation of 1,000 ng. Lavelle also discloses that a dose of 1,000 ng/mouse has a negative effect on the weight of the animals and affects their survival, i.e. is toxic.

Song et al (Song S K, Moldoveanu Z, Nguyen H N et al. 2007 Vaccine 25: 6359-6366) disclose the suitability of Korean mistletoe lectin as adjuvants that are administered mucosally.

However, plant-derived mistletoe lectins are inhomogeneous (Soler M H, Stoeva S, Schwamborn C et al. 1996 FEBS Letter 399: 153-157, Soler H S, Stoeva S, Voelter W 1998 Biochem Biophys Res Comm 246: 596-601) and are inconsistent or different from one another in terms of effect (EP 1051495 B1), and are not effective per se as an adjuvant or an immunomodulator. Therefore, although the Korean mistletoe lectin, for example, is associated with the RIP II proteins, this has strong structural differences in the structure and conformation compared to the recombinant mistletoe lectins under discussion here (Kang T B, Song S K, Yoon T J et al. 2007 J Biochem Mol Biol 40(6): 959-965). This disadvantage is that dosage cannot be adjusted exactly and plant-derived mistletoe lectins have impurities. In addition, plant-derived mistletoe lectins have differences in glycosylation that can affect the efficacy as an adjuvant (in particular kinetics, etc.).

Advantageously, the recombinant mistletoe lectins according to the invention do not have such glycosylation.

It is desirable to increase the immunogenicity of antigens by the use of adjuvants in order to obtain an efficient and improved immunological response in a host or patient. There is also a high demand for safe and effective adjuvants that increase the efficacy of vaccines and are easy to use.

The problem addressed by the present invention is that of providing a vaccine or adjuvant with which the immune response of a host or patient to antigens can be increased.

The problem is solved by providing a vaccine or adjuvant that contains a recombinant mistletoe lectin.

Surprisingly, it was found that recombinant mistletoe lectin can be used as a potential adjuvant when administered together with an antigen.

The invention therefore relates to an adjuvant containing at least one recombinant mistletoe lectin (referred to in the following as the adjuvant according to the invention).

The invention further relates to a vaccine containing an antigen together with an adjuvant according to the invention (referred to in the following as the vaccine according to the invention).

The invention therefore also relates to a drug or composition containing a vaccine according to the invention and, optionally, further auxiliary substances and additives and/or a pharmaceutically acceptable carrier or a pharmaceutically acceptable diluent.

The problem is solved by providing an adjuvant or a vaccine, wherein these contain recombinant mistletoe lectins, and the recombinant mistletoe lectins comprise the following amino acid sequences:

The drug according to the invention preferably comprises the mistletoe lectin A-chain (MLA) and the mistletoe lectin B-chain (MLB), either individually or in combination in either case, also in the form of dimers (see, for example, EP 0 751 221 or EP 1 051 495).

The recombinant mistletoe lectin polypeptide of the mistletoe lectin A-chain comprises the following sequences: SEQ ID No. 1-3, including the isoforms thereof or a functional fragment thereof.

The recombinant mistletoe lectin polypeptide of the mistletoe lectin B-chain comprises the following sequences: SEQ ID No. 4-12, including the isoforms thereof or a functional fragment thereof.

(referred to comprehensively in the following and above as “recombinant mistletoe lectins”)

Further, a recombinant mistletoe lectin according to the invention is preferred, a heterodimer comprising the sequences of SEQ ID No. 1 and SEQ ID No. 4; see, for example, EP 0 751 221, so-called Aviscumine, (see examples).

In the context of this invention, the expression “functional fragment” defines fragments of the stated polypeptides that have the same biological function as the polypeptide presented above comprising the particular amino acid sequence.

In this context, the expression “the same biological function” means, for example, that fragments or derivatives of the polypeptides induce the same signals in a cell as the stated peptides. Examples of fragments are peptide domains having defined functions. The “same biological function” also comprises the cytotoxicity, immunostimulation (of the native and the adaptive immune system), stimulation of the release of cytokines, antigenicity, the induction of expression or the activation of surface markers, the induction of apoptosis, or endorphin stimulation.

In this case, the expression “biological activity of the recombinant mistletoe lectin” refers to any biological activity from the spectrum of the totality of biological activities of recombinant mistletoe lectin. A function of this type is the pharmacological effect of recombinant mistletoe lectin, for example, in particular the suitability as an adjuvant in combination with an antigen (vaccine).

Investigations of ML-I monomers yielded 25 different isoforms, which result from different combinations of various A- and B-chains and different states of glycosylation of the chains.

With respect to the present invention, a mistletoe lectin polypeptide or a fragment thereof that comprises the sequence variability of the various MLA and MLB chains is therefore also considered, according to the invention, for the sequences of SEQ ID No. 1-12.

The drug according to the invention preferably contains at least one recombinant mistletoe lectin polypeptide comprising the sequences of SEQ ID No. 1-12 or a functional fragment thereof, or any combination thereof.

For example, an increased immune response was observed upon administration of a vaccine, according to the invention, with the hepatitis B surface antigen HBsAg (hepatitis B surface antigen), and with ovalbumin, as a “weak model antigen” (see examples). Furthermore, it was surprisingly found that the cell activation by recombinant mistletoe lectins takes place via an efficient and particularly advantageous activation of inflammasome comparable to the mechanism of action of aluminum (aluminum hydroxide).

In a further particularly advantageous embodiment of the invention, the dose to be administered to a patient (human or mammalian) is 5 to 600 ng/ml, in particular 250 to 450 ng/ml, 350 ng/ml, or 5 to 600 ng/patient, in particular 250 to 450 ng/patient, 350 ng/patient, of the recombinant mistletoe lectins, which, particularly preferably, is applied subcutaneously.

As shown in the examples, in the range of 5 to 50 ng/animal (or 50 to 500 ng/mL) or patient, an optimal interleukin-1β (IL-1β) release takes place in the narrow dosage range of 280-420 ng/patient (280-420 ng/mL), in particular after subcutaneous administration. At higher doses, interleukin is not released; rather, cytotoxic effects on healthy tissue are more likely to occur. IL-1β is a direct parameter for inflammasome activation, which is particularly advantageous for suitability as an adjuvant.

Particularly advantageously, homogeneous recombinant mistletoe lectins can therefore induce an optimized inflammasome activation. Due to the homogeneity of recombinant mistletoe lectin, an advantageous, exact dosage within a narrow dosage range is possible.

This also results in improved tolerability of the application, and therefore fewer applications are needed for a successful vaccination. Lavelle (supra) requires four applications for a successful vaccination, e.g. for lack of a sufficient inflammasome activation. According to example 2, as a reasonable analogy, a successful vaccination is achieved after only two applications.

The recombinant mistletoe lectins must be assigned to the adjuvant category “immune potentiators” (immune boosters (supra)) due to the mechanisms of action observed.

APCs are activated as follows:

1. Activation of phagocytes (dendritic cells and monocytes/macrophages) due to phagocytosis of Aviscumine with the consequences of
a. antigen presentation
b. initiation of T cell responses, and
c. cytokine secretion
   and
   2. Induction of apoptosis (e.g. monocytes/macrophages) with the consequences of
a. activation of adjacent phagocytes by the uptake of dead cells (e.g. apoptotic bodies) from apoptosis, and
b. cytokine secretion

Furthermore, advantageous inflammasome activation in monocytes by means of the recombinant mistletoe lectins according to the invention by caspase-1 stimulation and secretion of IL-1β and IL-18 is shown. A plurality of mechanisms for generating strong immune responses therefore characterizes recombinant mistletoe lectins as a strong and unique adjuvant.

In particular, the recombinant mistletoe lectins according to the invention allow a specific and significant inflammasome activation, which, according to the invention, is considered to be essential for suitability as an adjuvant.

The invention therefore also relates to the use of recombinant mistletoe lectins as an adjuvant in vaccines for strengthening the immune response of a host (animal, mammal, or human) to one or more antigens.

According to the invention, vaccines are provided, which contain at least one recombinant mistletoe lectin adjuvant and an antigen, e.g. an HBsAg antigen produced from yeast cells and, optionally, a pharmaceutically acceptable carrier or a pharmaceutically acceptable diluent, and, optionally, other components, such as sodium chloride, disodium phosphate dihydrate, sodium dihydrogen phosphate.

The vaccines according to the invention are preferably formulated in a phosphate-buffered saline solution and are provided as a sterile suspension for injection in a puncturable vial or a pre-filled syringe and trigger an immunological response in the host (animal, mammal, or human) or the donor/patient. The vaccines according to the invention induce the formation of specific T cells and the formation of humoral antibodies to HBsAg.

The vaccines according to the invention are used, e.g. for active immunization against hepatitis B viruses, caused by viruses of all known subtypes in non-immune persons of all ages. The populations to be immunized are defined in the official vaccination recommendations (Standing Committee on Vaccination at the Robert Koch Institute).

The following immunization schedules can be applied for a fundamental immunization with the vaccine according to the invention:

1. The immunization schedule calling for vaccinations in month 0, 1, 6 results in high antibody concentrations and, usually, optimal protection in month 7.
2. The accelerated immunization schedule calling for vaccinations after 0, 1 and 2 months enables a rapid buildup of protection due to inoculation. After 12 months, a fourth dose should be administered in order to induce long-term protection.

These immunization schedules, which are provided as examples, can be adjusted according to the national vaccination recommendations (Standing Committee on Vaccination at the Robert Koch Institute).

The vaccines according to the invention are preferably administered subcutaneously or intramuscularly.

The quantity of one or more antigens and a recombinant mistletoe lectin adjuvant in the vaccines according to the invention and the administered doses can be determined by means of techniques that are known to a person skilled in the art in the medical field. The type of dosing is determined by the treating physician in accordance with the clinical factors. A person skilled in the art knows that the type of dosing is dependent on various factors, such as the body height and weight, the body surface area, age, gender, or the general health of the patient, and on the preparation to be administered in particular, the duration and type of administration, and on other medications that may be administered in parallel.

Preferably, the recombinant mistletoe lectin adjuvant in the vaccines according to the invention are intended to be delivered as an aqueous, phosphate-buffered saline solution, and the antigen is usually present—depending on the unit—in an order of magnitude of nanograms, micrograms, up to milligrams (cf. examples provided below, for example).

The recombinant mistletoe lectin adjuvant according to the invention can be used with any antigen of interest, and therefore a vaccine according to the invention is provided.

Suitable antigens are preferably those that are used as potential vaccines and cannot be exclusively those, such as, e.g. HBsAg (supra), antigens against influenza viruses H1N1 and other subtypes, peptide antigens directed against immunogenic tumors, for example PAX peptides (Rodeberg D A, Nuss R A, Elsawa S F et al. 2006 Int J Cancer 119: 126-132, Yan M, Himoudi N, Pule M et al. 2008 Cancer Res 68(19): 8058-8065), and many others.

A preferred dosage of an adjuvant according to the invention is a concentration of 5 ng and higher.

Vaccines according to the invention are conveniently provided as liquid preparations or compositions that can be buffered to a selected pH value (supra).

The selection of suitable carriers and other additives depends on the desired mode of administration and on the type of the specific dosage form.

A pharmaceutically acceptable preservative can be used in order to increase the shelf life of the vaccines.

The vaccines according to the invention are prepared by mixing the components according to generally accepted methods, or by the individual administration thereof.

The following examples are intended to illustrate the invention without limiting the invention to these examples.

### EXAMPLES

### Example 1

### Activation of the Inflammasome by Aviscumine

PBMCs, isolated by FICOLL gradient purification from human blood from donors, are sown in titer plates and incubated with Avicusmine for 8 to 24 hours with or without LPS costimulation. LPS activates pro-IL-1β and pro-IL-18 via the Toll-like receptor 4 (TLR4) and subsequent caspase 1 activation without this leading to the release of cytokines by LPS alone.

In the co-culture of Aviscumine and LPS, the cytokines IL-1β and IL-18 are released in a manner dependent upon concentration. Secretion of IL-6 and TNF-alpha is not induced. (a 9-hour experiment). Aviscumine and LPS, each alone in the cell culture, do not induce the release of the cytokines IL-1β. Aviscumine alone is capable of releasing IL-18, and this effect is enhanced by LPS. If the PBMCs are freed from the monocytes by magnetic cell sorting (anti-CD14 MACS beads, Miltenyi), neither IL-1β nor IL-18 can be released in the remaining PBMCs by incubation with Aviscumine and LPS. The cytokines IL-1β and cells IL-18 are then released from the monocytes. The induction of IL-1β and IL-18 by incubating the PBMCs together with Aviscumine and LPS indicates the activation of the inflammasome complex.

In vitro, optimal IL-1β release from human peripheral blood mononucleated cells (PBMC) is seen in a narrow concentration range at an optimal concentration of 33 ng/mL, depending on the duration of incubation with Aviscumine in the absence of LPS. This effect follows a bell-shaped concentration-effect relationship (FIG. 8).

a.) Immunizations

Two immunizations are performed on a certain strain of inbred mice (BALB/c line), wherein Aviscumine, as the adjuvant, and the hepatitis B surface antigen (HBsAg) and—in a further experiment—ovalbumin, as the antigen, are administered together. The immunizations are performed at intervals of 3 weeks. The antigen-Aviscumine formulations are administered subcutaneously on day 1 and day 21, and the immunized animals are sacrificed 14 days after the last immunization.

In order to determine the immunogenicity, an intracelluar cytokine staining of antigen-specific CD8 and CD4 T cells and a determination of the antigen-specific antibodies is performed. This experimental arrangement makes it possible to detect a strengthened immune response, due to Aviscumine, at the cellular and humoral level.

### Example 2

### Immunization with Hepatitis B Surface Antigen (HBsAg)

The HBsAg antigen is a lipoprotein particle having a size of 20 nm. Such a virus-like particle is capable of inducing a humoral immune response, often even in the absence of an adjuvant. Mild HBsAg CD8 T cell responses can be stimulated in BALB/c mice by the antigen alone.

This combination of antigen and mouse strain makes it possible to analyze the capability of Aviscumine to strengthen mild humoral and cellular responses. In this case, AbISCO®-100 (ISCONOVA, Uppsala, Sweden) is used as the positive control, since it is known that this adjuvant increases the immune response in this model.

Mouse strain: BALB/C

2 subcutaneous immunizations (days 1, 21)

5 animals per group (3 animals for control groups 2 and 3); 31 animals in total

TABLE 1

| | HBsAg | Adjuvant | | |
|---|---|---|---|---|
| Group | (dose/animal) | (dose/animal) | | Remark |
| 1/A | 5 μg | AbISCO adjuvant | | positive control |
| 2/B | — | — | | PBS negative control |
| 3/C | — | 50 | ng Aviscumine | adjuvant control |
| 4/D | 5 μg | — | | antigen control |
| 5/E | 5 μg | 0.5 | ng Aviscumine | low dose |
| 6/F | 5 μg | 5 | ng Aviscumine | moderate dose |
| 7/G | 5 μg | 50 | ng Aviscumine | high dose |

The vaccine formulations are prepared fresh before each immunization. Aviscumine is diluted in PBS with 0.01% Tween 80.

### Reagents

5 μg recombinant HBsAg (Rhein Biotech GmbH, Düsseldorf, Germany) per animal is used at the antigen for formulations (100 μl volume per dose/animal).

As the control test, the adjuvant AbISCO-100 (Isconova, Uppsala, Sweden) is used, with 12 μg per dose. AbISCO-100 is an adjuvant that has been optimized for use in mice.

The following synthetic peptides are used for BALB/c mice to restimulate CD8+ cells: HBsAg₂₈₋₉ IPQSLDSWWTSL as MHC class I (L^{d})-restricted, HBsAg-specific peptides. Malaria CSP₂₈₀₋₂₈₉ SYVPSAEQI as MHC class I (K^{d})-restricted, irrelevant control peptides.

CD4+ specific T cells are restimulated with HbsAg antigens (used as for the vaccine formulation).

### Investigations

a.) Determination of HbsAg-Specific, IFNγ-Producing CD8+ T Cells from the Spleen

On day 14 after the second immunization, a single-cell suspension of the spleen is restimulated ex vivo for 4 hours with the corresponding antigen-specific peptides. Brefeldin A is added in order to keep the cytokines produced intracellular. The cell surfaces are stained for CD8, fixed, and permeabilized for subsequent intracellular IFN-γ staining. Stained cells are analyzed with a Beckmann Coulter flow cytometer (FC 500) using CXP software. 60 000 CD8 positive cells are analyzed. The number of CD8+IFN-γ+ T cells per 10⁵ CD8+ T cells is indicated.

b.) Determination of HbsAg-Specific, IFNγ-Producing CD4+ T Cells from the Spleen

On day 14 after the second immunization, a single-cell suspension of the spleen is restimulated ex vivo overnight with 1 μg/mL recombinant HBsAg. The medium for restimulation is used as the negative control. After treatment with Brefeldin A for 4 hours, the cell surfaces are stained for CD4, fixed, and permeabilized for subsequent intracellular IFN-γ staining. The cells are analyzed with a Beckmann Coulter flow cytometer (FC 500) using CXP software. 60 000 CD4 positive cells are analyzed. The number of CD4+IFN-γ+ T cells per 10⁵ CD4+ T cells is indicated.

c.) Quantification of Specific HBV Surface Antibodies in Mouse Serum

IMx AUSAB (ELISA) with the IMx Reader (Abbott Diagnostics) is used in order to quantify anti-HBsAg antibodies. The test is carried out in accordance with the manufacturer's recommendations.

d.) Determination of the T-Helper Cell (Th) Profile of the Immune Response

Antigen-specific IgG1 and IgG2b titers are determined by ELISA and the IgG1/IgG2b ratio is used to evaluate the Th profile.

### Statistics

The t test is used (GraphPad Prism 5 software) to perform the statistical analysis of the differences between two groups.

### Results

### HbsAq-Specific T Cell Response:

After 2 immunizations, an increased HBsAg-specific CD8+ T cell response was observed in the 50 ng and 5 ng Aviscumine groups (see FIG. 4).

The group having HBsAg with 0.5 ng Aviscumine did not exhibit a strengthened CD8+ response, compared to HBsAg alone (which has a certain CD8+ response in BALB/c mice). As expected, the positive control AbISCO exhibited an effective CD8+ response. A background was not observed in the buffer and the adjuvant control groups (see FIG. 4).

A HBsAg-specific CD4 T cell response was observed in the group that received HBsAg+50 ng Aviscumine (see FIG. 5).

The strength of this CD4+ response is comparable to that obtained with AbISCO. Lower doses of Aviscumine as well as buffer and adjuvant control did not induce an HBsAg-specific CD4 response (see FIG. 5).

### Anti-HBsAg Antibody Response

A weak anti-HBs antibody response was observed 2 weeks after the second immunization in the 50 ng Aviscumine group (see FIG. 6).

A weak anti-HBs antibody response is observed in BALB/c mice even with repeated immunization with HBsAg alone.

As expected, the positive control AbISCO induces a strong antibody response, while the buffer and adjuvant control are negative.

The T helper cell profile was not determined for HBsAg-immunized animals due to low or negative anti-HBs titers.

### Example 3

### Immunization with Ovalbumin (OVA)

Ovalbumin (OVA) is a soluble, monomeric protein which can induce anti-OVA antibodies only in combination with adjuvants. AbISCO®-100 (ISCONOVA, Uppsala, Sweden) is used as the positive control, since it is known that this adjuvant increases the immune response in this model.

Mouse strain: C57BL/6

2 subcutaneous immunizations (days 1, 21)

5 animals per group (3 animals for control groups 9 and 10); 31 animals in total

| Group | OVA (dose/animal) | Adjuvant (dose/animal) | | Remark |
|---|---|---|---|---|
| 8/H | 10 μg | AbISCO adjuvant | | positive control |
| 9/ I | — | — | | PBS negative |
| | | | | control |
| 10/K | — | 50 | ng Aviscumine | adjuvant control |
| 11/L | 10 μg | — | | antigen control |
| 12/M | 10 μg | 0.5 | ng Aviscumine | low dose |
| 13/N | 10 μg | 5 | ng Aviscumine | moderate dose |
| 14/0 | 10 μg | 50 | ng Aviscumine | high dose |

The vaccine formulations are prepared fresh before each immunization. Aviscumine is diluted in PBS with 0.01% Tween 80.

### Reagents

10 μg ovalbumin, endotoxin-poor, (Hyglos GmbH, Regensburg, Germany) per animal is used as the antigen for formulations (100 μl volume per dose/animal).

As the control test, the adjuvant AbISCO-100 (Isconova, Uppsala, Sweden) is used, with 12 μg per dose. AbISCO-100 is an adjuvant that was optimized for use in mice.

### Investigations

a.) Quantification of Specific OVA Antibodies in Alpha Diagnostics International Mouse Serum

The mouse anti-OVA ELISA from Alpha Diagnostics International is used to quantify anti-OVA antibodies. The test is carried out in accordance with the manufacturer's recommendations.

b.) Determination of the T Helper Cell (Th) Profile of the Immune Response

Antigen-specific IgG1 and IgG2b titers are determined by ELISA and the IgG1/IgG2b ratio is used to evaluate the Th profile.

### Statistics

The t test is used (GraphPad Prism 5 software) to perform the statistical analysis of the differences between two groups.

### Results

### Anti-OVA Antibodies

An OVA-specific antibody response was observed in the group with 50 ng Aviscumine (see FIG. 7). The buffer and the adjuvant control groups were negative. The AbISCO control group was positive (see FIG. 7).

The determination of OVA-specific IgG1 and IgG2b isotypes in the highest Aviscumine groups (50 ng) showed that the anti-OVA antibodies were dominated by IgG1. This is an indication of a Th2 profile of the immune response.

### General Remark

After the first and second immunizations, no adverse events were observed in the animals. All animals were healthy at the time of sacrifice.

### Conclusion

The investigation of Aviscumine with the antigens HBsAg and OVA showed, in mice, after subcutaneous immunizations (on days 1 and 21), an adjuvant effect at higher administered doses (5 (50 ng/mL) and 50 ng (500 ng/mL)).

### T Cell Response:

A CD8+ and a CD4+ response to HBsAg is observed when this is administered together with Aviscumine (50 ng (500 ng/mL) and 5 ng (50 ng/mL) dose). In particular, the CD4+ T cell response is strongly supported by Aviscumine.

Since HBsAg alone exhibits a certain CD8+ response in BALB/c mice, as is known, the adjuvant effect of Aviscumine strengthens this baseline level.

These results show that Aviscumine has adjuvant properties, in particular to CD4+ T helper cell responses.

### Antibody Response

An anti-HBsAg response was negative except in the case of one animal out of 5 animals. It is known that anti-HBsAg antibodies have slow kinetics. Therefore, it is likely that the animals that exhibited no anti-HBsAg antibodies would have become positive at a later point in time. Ideally, antibodies are determined 4 weeks after the immunization. However, the investigation was focused on the T cell response and therefore the animals had to be sacrificed 14 days after the immunization.

An anti-OVA antibody response was observed at the time the animals were sacrificed, i.e. 14 days after the second immunization.

The determination of the anti-OVA antibody isotypes exhibited a Th2 profile of the immune response. This is consistent with the induction of CD4 T cells that was observed.

### Summary of the Results

Aviscumine doses of 50 ng (500 ng/mL) and 5 ng (50 ng/mL) per animal strengthened specific CD8 and, in particular, CD 4 T cell responses to HBsAg, which is a complex lipoprotein antigen. Specific antibodies having a Th2 profile against OVA were determined.

These data show that Aviscumine has an adjuvant effect with a strengthened CD4 T helper cell response.

The doses of Aviscumine that exhibited a strengthened immune response to HBsAG were low.

The adjuvant effect of Aviscumine can probably be strengthened by modification of the doses or the booster immunizations or depot formulations in order to achieve specific antibody concentrations in every case.

### DESCRIPTION OF THE FIGURES

FIG. 1 describes the mechanism of action of non-specific adjuvants (figure from Lamine Mbow M et al. 2010 (supra)).

FIG. 2 shows the secretion of IL-1β (pg/ml) from human PBMCs by Aviscumine and LPS.

FIG. 3 shows the secretion of IL-18 (pg/ml) from human PBMCs by Aviscumine and by Aviscumine and LPS.

FIG. 4 shows the results of the investigation of the HBsAg-specific T cell response, wherein the CD8 T cell response was measured in BALB/c mice. Restimulation took place with HB surface epitope 28-39.

FIG. 5 shows the results of the investigation of the HBsAg-specific T cell response, wherein the CD4 T cell response was measured in BALB/c mice. Restimulation took place with HBsAg.

FIG. 6 shows the results of the investigation of the anti-HBs antibody response in BALB/c mice.

FIG. 7 shows the results of the investigation of the anti-OVA antibody response in C57BL/6 mice.

FIG. 8 shows the Aviscumine-induced IL-1β secretion (pg/mL) from peripheral blood mononucleated cells (PBMC) from voluntary donors after incubation for 24 hours in the presence of LPS.

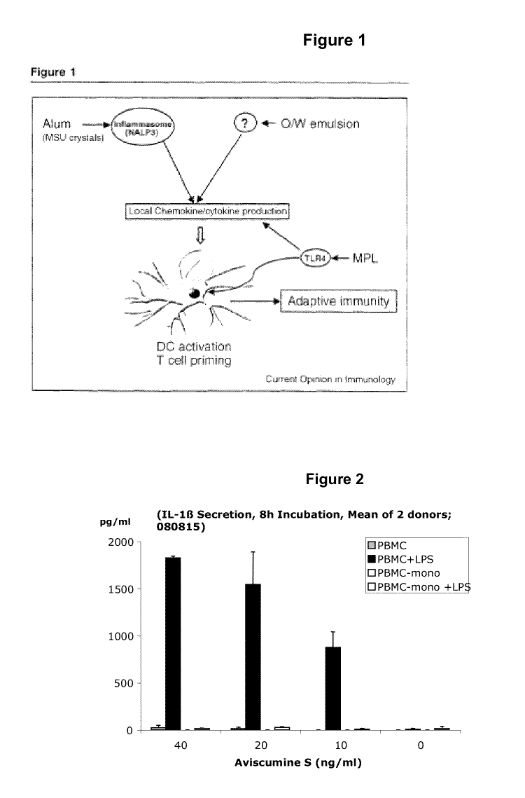

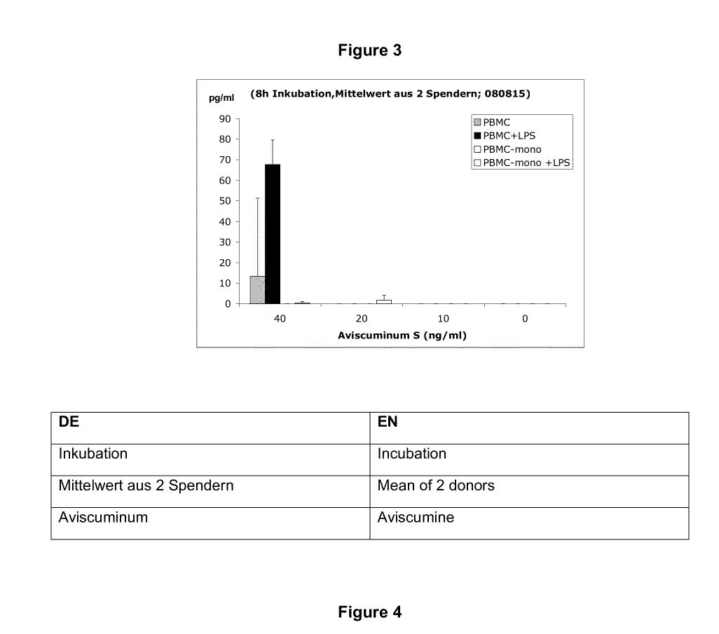

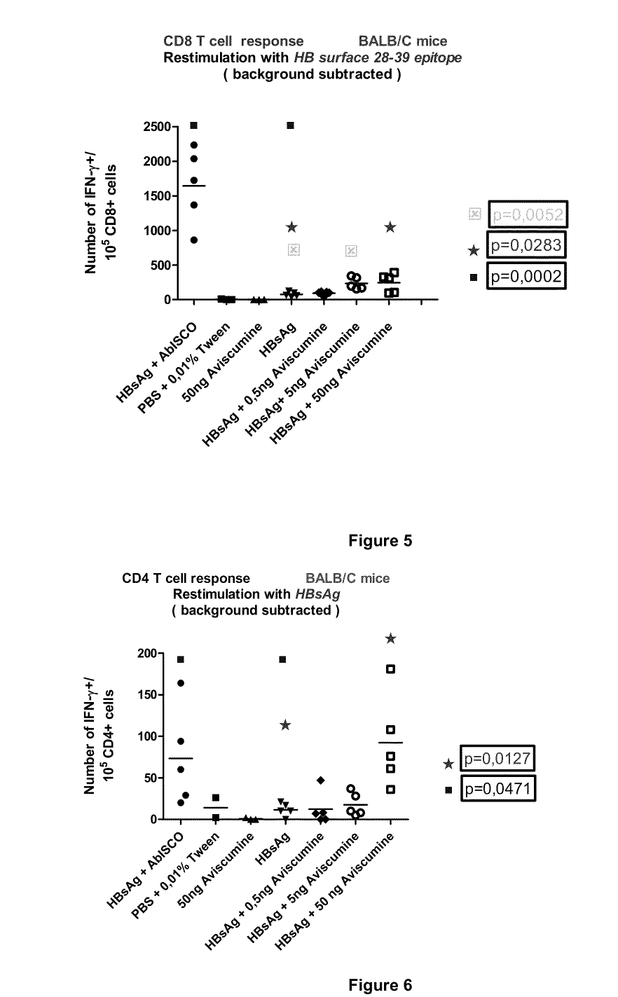

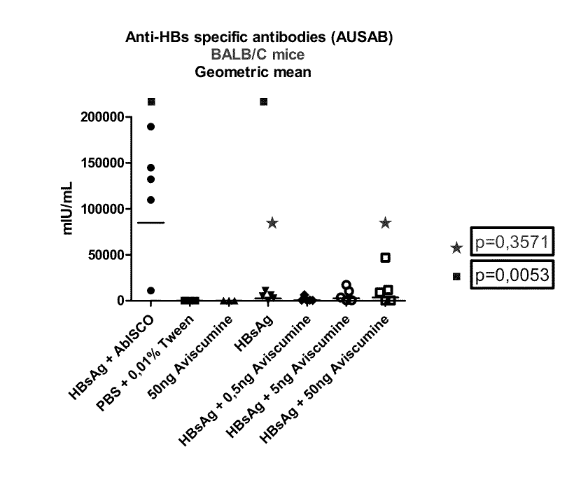

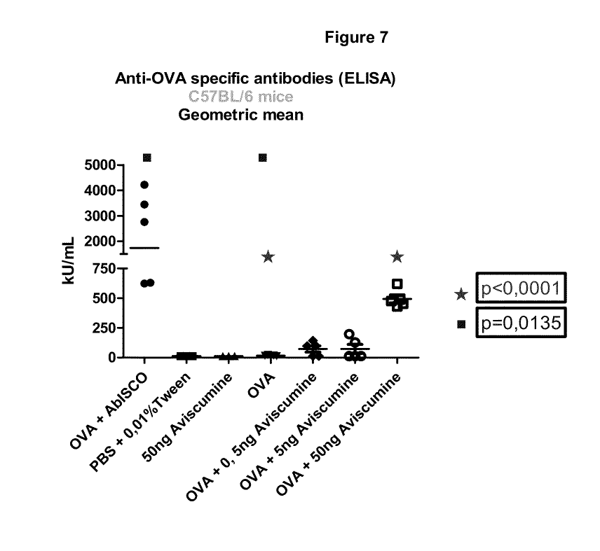

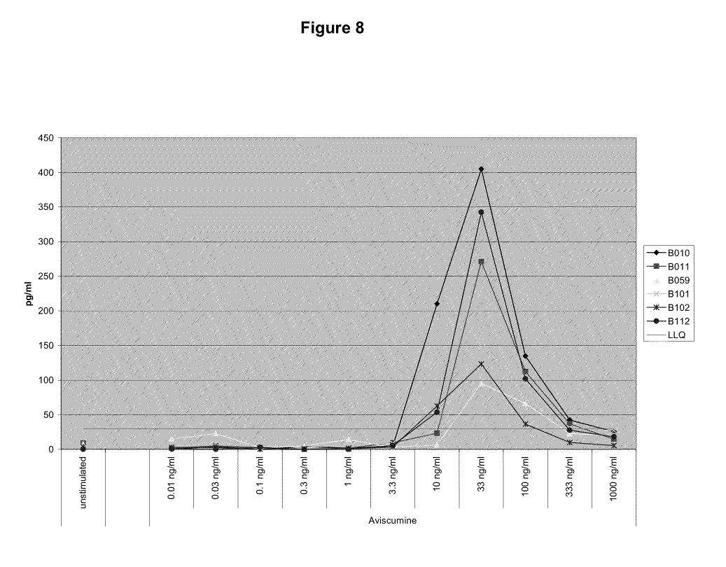

1. A vaccine containing one or more antigens and at least one adjuvant selected from the group consisting of recombinant mistletoe lectins comprising the amino acid sequences of SEQ ID NO: 1 and/or SEQ ID NO: 4.

2. A drug or composition containing one or more antigens and at least one adjuvant selected from the group consisting of recombinant mistletoe lectins comprising the amino acid sequences of SEQ ID NO: 1-12, or parts and fragments thereof, or a combination thereof for use as a vaccine, wherein the adjuvant is present in a concentration of 5 to 600 ng/ml, 250 to 450 ng/ml, 350 ng/ml, or 5 to 600 ng/patient, 250 to 450 ng/patient, 350 ng/patient.

3. The vaccine of claim 1, wherein said vaccine is administered subcutaneously or intramuscularly.

4. The drug or composition according to claim 2, wherein the recombinant mistletoe lectin is a mistletoe lectin A-chain selected from the group consisting of amino acid sequences of SEQ ID NO: 1-3, or comprises parts and fragments thereof, or is a combination thereof.

5. The drug or composition according to claim 2, wherein the recombinant mistletoe lectin is a mistletoe lectin B-chain selected from the group consisting of amino acid sequences of SEQ ID NO: 4-12, or comprises parts and fragments thereof, or is a combination thereof.

6. The vaccine of claim 1, wherein the vaccine also contains a pharmaceutically acceptable carrier or a pharmaceutically acceptable diluent, optionally auxiliary substances and additives.

7. A drug or a composition containing the vaccine according to claim 1.

8. The drug or composition of claim 2, wherein said drug or composition is administered subcutaneously or intramuscularly.

9. The drug or composition of claim 2, further comprising a pharmaceutically acceptable carrier or a pharmaceutically acceptable diluent, optionally auxiliary substances and additives.
